(19) United States
(12) Patent Application Publication
(10) Pub. No.: US 2018/0168721 A1
(43) Pub. Date: Jun. 21, 2018

(54) Non-Occlusive Circumferential Vascular Ablation Device

(71) Applicant: CORFIGO, INC., Montclair, NJ (US)

(72) Inventor: Jason Sperling, Cherry Hills Village, CO (US)

(73) Assignee: Persistent AFIB Solutions, Inc., Montclair, NJ (US)

(21) Appl. No.: 15/572,420

(22) PCT Filed: May 6, 2016
(86) PCT No.: PCT/US16/31286

§ 371 Date: Nov. 7, 2017

Related U.S. Application Data
(60) Provisional application No. 62/158,037, filed on May 7, 2015.

(51) Int. Cl. A61B 18/14; A61B 5/00; A61B 5/0215; A61B 5/042; A61B 5/06; A61B 18/02

(52) U.S. Cl. CPC A61B 18/1492; A61B 5/6853; A61B 5/0215; A61B 2017/00084; A61B 5/065; A61B 5/4848; A61B 18/0206; A61B 5/0422

(57) ABSTRACT

A device for internal circumferential ablation of a tubular vascular structure includes an expandable structure having a central opening extending therethrough to allow a relatively unobstructed flow of body fluid through the tubular vascular structure. The expandable structure is expandable from a collapsed configuration to an expanded configuration, with the expandable structure being configured to be secured in a desired location on the tubular vascular structure. The expandable structure further is configured to emit or absorb energy to ablate tissue. A method of circumferential ablation of a terminal segment of a coronary sinus or other vascular structure using a catheter system in order to treat heart rhythm disorders and performing ablation without significant obstruction of blood flow is further disclosed.

### CROSS-REFERENCE TO RELATED APPLICATION

This application claims priority to International PCT Application No. PCT/US16/31286, filed on May 6, 2016, which claims priority to U.S. Provisional Patent Application No. 62/158,037 filed May 7, 2015, entitled DOUBLE-BALLOON ABLATION/COUNTER-ABLATION DEVICE, which is incorporated by reference herein in its entirety.

### BACKGROUND OF THE DISCLOSURE

Atrial fibrillation (AFib) is a common heart rhythm disturbance seen in clinical practice. AFib is a morbid condition that leads to symptoms of fatigue, low energy, and short-windedness with exertion and palpitations (irregular heart beat). AFib is a major cause of stroke, heart failure and hospitalizations. It is estimated that upwards of 6-8 million people in the United States alone have this chronic condition, and its prevalence is increasing as the world population ages. The treatment of AFib is a major source of healthcare spending. The most effective treatments that have emerged for this condition have been “ablation” treatments that eliminate the electrical capabilities of relevant areas of the left (and sometimes right) atrium (upper heart chamber(s)). The most consistent target area that is ablated is pulmonary veins, which are ablated as they join the left atrium from the lungs. Energy sources such as radiofrequency, laser, ultrasound and most recently cryothermy have been effective to different degrees. Pulmonary vein ablation (or isolation) has been more effective for early stage AFib (paroxysmal or intermittent) than for later stage AFib (persistent or chronic). For the latter, additional ablation is required to terminate AFib, with most newer treatments targeting the back (posterior) wall of the left atrium, which adds a degree of invasiveness but also confers much better efficacy, but not 100%. In addition, extensive treatment of the left atrium sometimes unmasks other morbid dysrhythmias, such as atrial flutters or tachycardias that may be very difficult to treat.

Electro-anatomically, normal (“sinus”) rhythm is controlled by two “nodes,” the first (sino-atrial) located high within the right atrium heart chamber, and the second (atrio-ventricular) located at the junction between the right atrium and right ventricle (lower) chambers. Electrical impulses travel from one node to the next along conduction bundles located within the wall (septum) between the atrium (upper) heart chambers. Other conduction bundles exist in predictable locations in order to spread the electrical impulses to the other areas of the heart. In AFib, abnormal electrical voltage has developed (most often within the left atrium), which may be stronger than the SA node voltage, and takes advantage of these conduction bundles to “hijack” the rhythm by its ability to reach the second AV node, faster than the SA node voltage can get there.

There are several reasons why the left atrial voltage is able to out-compete the SA node. These reasons may include, in no particular order—1) left atrial voltage that has developed may far exceed the voltage that can be generated by the SA node, 2) anatomically, portions of the left atrium are much closer to the AV node than the SA node (originates high within the right atrium), 3) the left atrium uses a very special and fast conduction bundle/structure called the coronary sinus, which essentially terminates in the AV node, and 4) in advanced forms of AFib, the back wall of the left atrium is a highly (electrically) conductive structure that conducts aberrant voltage very efficiently to the coronary sinus.

The coronary sinus is a large vein that drains blood that has traveled through the heart muscle itself. The coronary sinus “dumps” all of that blood into the right atrium, just below the AV node. The wall of the vein is muscular and these smooth muscle cells conduct electricity very well. While ablation within the coronary sinus can be performed, this is usually done as “touch-up” work where residual abnormal electrical foci are found residually after a more extensive ablation procedure. It would be useful to be able to ablate the termination of the coronary sinus into the right atrium, but this would be difficult to achieve due to the eccentric shape of the coronary sinus opening (“os”) but also especially because of its proximity to the AV node itself. Ablation too close to the AV node will result in “heart block,” which will require permanent pacemaker implantation, which is a morbid medical condition. Ablation of the coronary sinus during open-heart surgery has been shown to improve the efficacy of procedures performed to cure AFib, but again without 100% efficacy and also with an increased incidence of left atrial flutter rhythms that emerge and likely use the remaining (non-ablated) portions of the coronary sinus. Therefore, ablation even just a short distance away from the sinus opening itself will still allow electrical signals to enter the terminal portion of the coronary sinus and thus decrease effectiveness in controlling AFib and left atrial flutter.

A device has been conceived that will allow for thorough coronary sinus ablation at the level of the sinus opening, while protecting the AV node from ablation injury.

### SUMMARY OF THE DISCLOSURE

One aspect of the present disclosure is directed to a device for internal circumferential ablation of a tubular vascular structure. In one embodiment, the device comprises an expandable structure having a central opening extending therethrough to allow a relatively unobstructed flow of body fluid through the tubular vascular structure. The expandable structure is expandable from a collapsed configuration to an expanded configuration, with the expandable structure being configured to be secured in a desired location on the tubular vascular structure. The expandable structure further is configured to emit or absorb energy to ablate tissue.

Embodiments of the device further may include configuring the expandable structure as a balloon, which includes an ablating element. The balloon may be generally shaped like a torus or elongated cylinder having a channel. The balloon may include a surface having a plurality of sensors. The expandable structure may be configured to apply a radial contact force on the tubular vascular structure to secure the expandable structure to the tubular vascular structure. The balloon may be positioned at a tip of a longer catheter. The catheter may be used to deliver gas or liquid to and from the balloon to accomplish ablation by releasing or absorbing energy. The balloon may house an array of small bore tubes to generally equally disperse gas or liquid for equal ablation along an expanded circumference of the balloon. The balloon may be a compliant balloon that generally assumes a shape of the vascular structure within which it expanded. The device further may comprise a second expandable structure adjacent to the balloon. The second expandable structure may include a larger surface area than the diameter of the balloon. The expandable structure may include a thermally conductive stent that conforms to a shape of the tubular vascular structure, with the stent being connected to either a heating element or a cooling element to conduct energy through the stent. The device further may comprise surface sensors provided on the expandable structure. The device further may comprise a second expandable structure adjacent to the thermally conductive stent, the second expandable structure including a larger diameter than the thermally conductive stent. The expandable structure may include a stent that conforms to a shape of the tubular vascular structure, with the stent housing and centering an ablating element in its center. The ablating element may be omnidirectional and ablates using ultrasound, microwave, laser, or another form of energy. The device further may comprise surface sensors provided on the expandable structure.

Another aspect of the present disclosure is directed to a method of circumferential ablation of the terminal segment of the coronary sinus or other vascular structure using a catheter system in order to treat heart rhythm disorders and performing the ablation without significant obstruction of blood flow. In one embodiment, the method comprises: positioning an expandable structure of the catheter system into a tubular vascular structure, the expandable structure having a central opening extending therethrough to allow a relatively unobstructed flow of body fluid through the tubular vascular structure; securing the expandable structure in a desired location on the tubular vascular structure; and expanding the expandable structure from a collapsed configuration to an expanded configuration.

Embodiments of the method further may include configuring the expandable structure as a balloon including an ablating element. The balloon may be generally shaped like a torus or elongated cylinder having a channel. The method further may include sensing one of voltage, temperature and pressure with wiring conducted along the catheter system. The balloon may include a surface having a plurality of sensors. Securing the expandable structure may include expanding the expandable structure to apply a radial contact force. The method further may include delivering gas or liquid to and from a catheter in fluid communication with the balloon.

### BRIEF DESCRIPTION OF THE DRAWINGS

Various aspects of at least one embodiment are discussed below with reference to the accompanying figures, which are not intended to be drawn to scale. Where technical features in the figures, detailed description or any claim are followed by references signs, the reference signs have been included for the sole purpose of increasing the intelligibility of the figures, detailed description, and claims. Accordingly, neither the reference signs nor their absence are intended to have any limiting effect on the scope of any claim elements. In the figures, each identical or nearly identical component that is illustrated in various figures is represented by a like numeral. For purposes of clarity, not every component may be labeled in every figure. The figures are provided for the purposes of illustration and explanation and are not intended as a definition of the limits of the disclosure. In the figures:

FIGS. 1 A-E are schematic views of a catheter having a device of an embodiment of the present disclosure;

FIGS. 2A-D are schematic views of a catheter having a device of another embodiment of the present disclosure;

FIGS. 3A-D are schematic views of a catheter having a device of another embodiment of the present disclosure; and

FIGS. 4A-C are schematic views of a catheter having a device of another embodiment of the present disclosure.

### DETAILED DESCRIPTION OF THE PREFERRED EMBODIMENTS

A device comprises a system that can engage the coronary sinus opening and hold the ablation system in an exact, proper position so that ablation of a generally cylindrical zone of the terminal portion of the coronary sinus can be carried out using an energy source. The device includes a substantial central void to allow fluid (blood) to flow through itself either by a central hollow core or other design feature to prevent occlusion or obstruction of the coronary sinus during the ablation. The surface of the atrio-ventricular junction in the vicinity of the coronary sinus may be protected by a counter-ablation mechanism using either warming or cooling.

In one embodiment, the coronary sinus is entered using standard established trans-femoral techniques, with a guide-wire. Over this guide-wire, a catheter/balloon or stent system is advanced retrograde into the coronary sinus, beyond its os (opening).

In one embodiment, a device includes a conformable balloon that is expanded within the terminal portion of the coronary sinus. The expandable balloon forms the shape of a torus or elongated torus, whose surface area is in contact with the interior of the terminal coronary sinus and conforms to its tapering cylindrical shape. The expandable balloon also has a sizeable central core or void to allow blood to pass through the balloon from the proximate portion of the coronary sinus without significant build-up of pressure in the partially-obstructed proximate coronary sinus. In order to assure correct position within the terminal segment of the coronary sinus, the expandable torus-shaped balloon has surface sensors to detect voltage. These sensors are used to create a voltage map that shows which portions of the balloon are in contact with the terminal coronary sinus. The general idea is that most of the ablating balloon will be housed within the coronary sinus when expanded, with a small portion residing within the right atrium, to assure that the ablating balloon is correctly positioned within the actual junction between the coronary sinus and the right atrium, and not deeper within the coronary sinus, which would be less desirable. The balloon will be configured to circumferentially ablate the contacted portions of the coronary sinus after inflation. Energy sources such as radiofrequency, laser, microwave, ultrasound and others are considered, but a likely iteration will utilize cryothermy. An appropriate gas such as Argon, Nitrous Oxide or other could be passed through a throttle or capillary tube or any other mechanism possibly using the Joule-Thompson effect to create very cold temperatures within the ablating balloon, while still allowing the balloon to conform to the shape of the coronary sinus and maintain contact and also maintain sufficiently low balloon pressure so as to avoid injury to the coronary sinus or the heart. It is anticipated that a system will be created to allow for more or less equal dispersion of the cold gas within the balloon. This can be accomplished by an array of small bore tubes within the balloon, and the balloon would be expected to have an entry and exit port for the gas. After ablation is completed, a second gas can be used for a warming effect (such as Helium) to facilitate thawing of the balloon so it can be safely deflated and pulled away from the wall of the coronary sinus. The entire catheter system can then be removed from the heart and venous system.

It is anticipated that this type of ablation system can also be used to ablate other tubular-type structures, for instance the superior or inferior vena cava or even the base of the left atrial appendage.

Another design can utilize an expandable stent that conforms to the shape of the terminal coronary sinus, and ablate the wall of the sinus in a circumferential fashion while allowing blood to pass through unimpeded. The stent can be connected to a heating element or a cooling element that causes cryoablation of the contacted coronary sinus by conduction. Surface sensors can be used to detect voltage and temperature. Voltage sensors may be used to assure correct positioning of the ablating stent, and also could detect acute success of the procedure. The stent would be collapsed and recaptured for removal from the heart and body.

Embodiments of the device may include another expandable device (stent or balloon or other) that lies immediately adjacent to the ablating structure, and whose purpose is to assure proper placement of the ablating structure within the terminating portion of the coronary sinus by acting as a “stopper” that contacts the right atrium surrounding the sinus opening (terminating rim) of the coronary sinus. Another purpose of the second adjacent expandable seating structure can be to carry out counter-ablation by either warming or heating to prevent unintended ablation of structures that surround the coronary sinus.

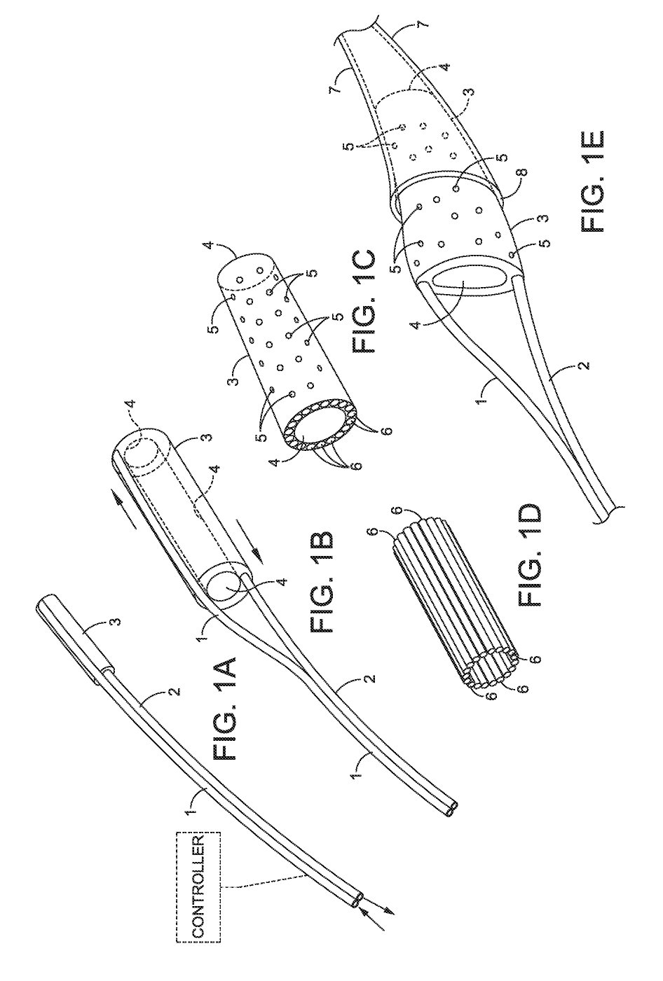

Referring to the drawings, and more particularly to FIGS. 1A-1E, a catheter system has a tip in the form of an expandable balloon in the general shape of an elongated torus or cylinder, with a generous central core to allow blood to pass through the expandable balloon in a minimally obstructive fashion while the balloon carries out ablation of the blood vessel wall within which it is deployed or expanded. The balloon may include an internal tube system or array to facilitate equal dispersion of the ablating gas or liquid. Surface sensors detect, for example, voltage, temperature, and pressure, to facilitate the ablation procedure, and connect to a controller system for electronic display, quality control and gas delivery/exhaust.

In FIG. 1A, a catheter system includes an inlet tube 1 for delivering gas or liquid (not shown) into an ablating expandable balloon 3, and an outlet or exhaust tube 2 for recycling of gas or liquid and control of pressure within expandable balloon 3 once expanded (not shown). The trailing edge of the ablation catheter is connected to an off-table controller to handle gases and electronics.

In FIG. 1B, the expandable balloon 3 of the catheter system assumes the general shape of an elongated torus or cylinder including a larger central core 4 that allows passage of blood through the expandable balloon 3 while the expandable balloon is positioned within a blood vessel (not shown) targeted for ablation. The inlet tube 1 and the outlet/exhaust tube 2 are embedded within expandable balloon 3 for gas or liquid exchange within the expandable balloon.

In FIG. 1C, the expandable balloon 3 is shown unattached to the inlet tube and the outlet/exhaust 2 and again visualized is the large central core 4. Also shown are surface sensors 5, which may detect any one of voltage, temperature and pressure with wiring conducted along the catheter system (not shown). Also, in this embodiment, the expandable balloon 3 includes a micro-tube array 6, which can be used to facilitate symmetrical gas or liquid dispersion within the expandable balloon to promote symmetrical circumferential ablation.

In FIG. 1D, the catheter system includes a micro-tube array 6 that is configured to reside within the expandable balloon 3 (not shown in FIG. 1D) and spread out along the perimeter of the expandable balloon when expanded. The micro-tube array 6 promotes symmetric dispersion of gas or liquid used for ablation.

In FIG. 1E, the expandable balloon 3 is shown expanded within a blood vessel 7, specifically, the coronary sinus. The expandable balloon 3 straddles a termination or sinus opening (os) 8 of the coronary sinus 7, with portions of the expandable balloon 3 touching the interior wall of the coronary sinus 7 and its sinus opening 8, and other portions not positioned within the coronary sinus at all. The expandable balloon 3 generally conforms to the shape of coronary sinus 7. Again seen are surface sensors 5, the large central core 4, and the inlet tube 1 and the outlet/exhaust tube 2 for gas or liquid conveyance to and from the balloon 3.

In one embodiment, an appropriate gas, such as Nitrous Oxide (N₂O) or Argon, can be delivered under high pressure via inlet tube 1 of the catheter system. As the pressure drops in the expandable balloon 3 and the gas expands within the expandable balloon, the gas cools according to the well-known scientific principle called the Joule-Thompson effect, causing very low temperature in expandable balloon 3, which causes cryoadhesion and fixation of expandable balloon 3 to the interior contacted wall of the blood vessel 7. While cryoablation of the vessel 7 is then carried out, blood continues to pass relatively unobstructed through large central core 4.

The outlet/exhaust tube 2 of the catheter system, which has a larger diameter than the inlet tube 1, allows the gas to escape and for pressure within the expandable balloon 3 to be controlled. A plurality of surface sensors 5 are connected along the catheter system to a controller/console (FIG. 1A). The surface sensors 5 (e.g., voltage sensors) facilitate proper positioning of the expandable balloon to straddle the termination or sinus opening 8 of the coronary sinus. After ablation is completed, the ablation expandable balloon 3 is deflated and the catheter system withdrawn from the coronary sinus.

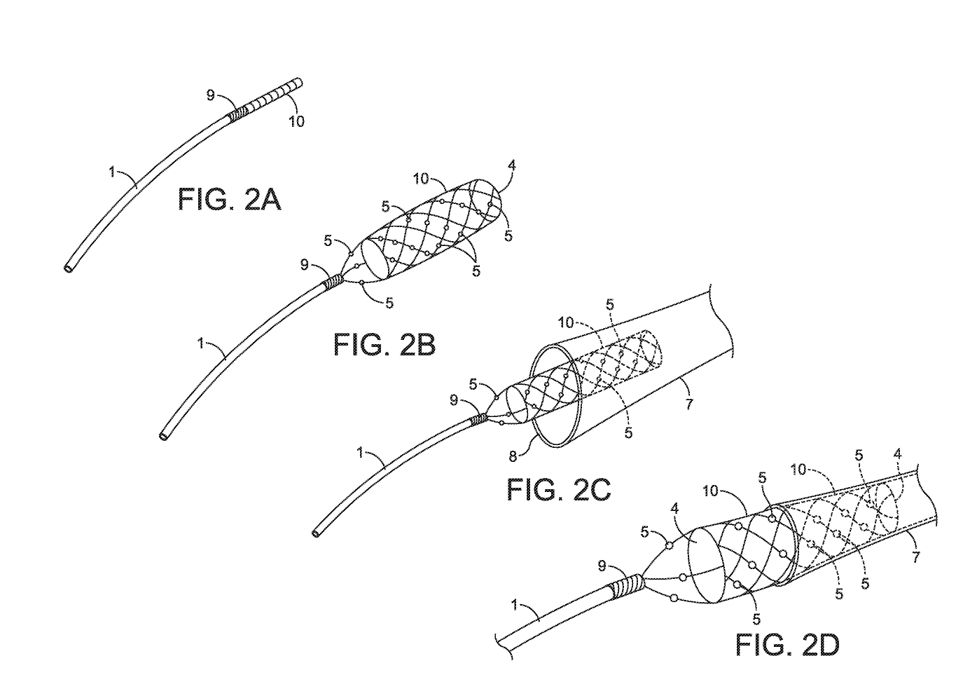

Referring to FIGS. 2A-D, a catheter has a tip in the form of an expandable thermally conductive stent, powered by an energy source connected to the stent. Surface sensors detect, for example, voltage, temperature, and pressure, to facilitate the procedure. The catheter is passed into the opening of the target blood vessel to be ablated and expanded into contact with the internal vessel wall. The tip of the catheter is collapsed and retrieved (not shown) after the ablation is completed.

In FIG. 2A, a catheter system includes the inlet tube 1 having a terminal end 9, which is an element to deliver or resorb energy, and is connected to an expandable/collapsible thermally conductive stent 10. In one embodiment, the element can be a heating element or a cooling element connected to the thermally conductive stent.

In FIG. 2B, in a particular embodiment, the inlet tube 1 of the ablating catheter includes a terminal end 9 connected to the stent 10. Because of the nature of the wire-material stent 10, there will be free-flow of blood through the stent, depicted by opening 4. The surface sensors 5 transmit information via wiring along inlet tube 1.

In FIG. 2C, passage of the ablating catheter system into a target vessel is shown with the partially deployed stent 10 within the coronary sinus 7. Also shown is the desired positioning of the ablating stent straddling the termination or sinus opening 8 of the coronary sinus 7. As previously shown, the device includes a plurality of surface sensors 5 and the terminal end 9 that conducts or resorbs energy by its connections with stent 10.

In FIG. 2D, the thermally conductive ablating stent 10 is deployed within the coronary sinus 7, and straddles the coronary sinus termination or sinus opening 8. The stent 10 generally conforms to the shape of the coronary sinus 7. Ablation energy or cryothermy is conducted via the inlet tube 1 of the catheter, and dispensed via the terminal end 9, which is directly connected to the ablating stent 10. Stent 10 is an expandable/collapsible stent comprised of highly thermally conductive metal or alloy that delivers or resorbs energy generally by conduction and ablates the contacted interior wall of the blood vessel within which it is deployed. The surface sensors 5 facilitate correct positioning, straddling the coronary sinus opening 8, and the ablating stent 10 is temporarily fixed in position by both radial tension and cryoadhesion, for example, if the ablation source is cryothermy. Blood can flow freely through the interior of the ablating stent 10, depicted by large flow channel 4. After ablation is complete, the stent 10 is re-collapsed and withdrawn (not shown).

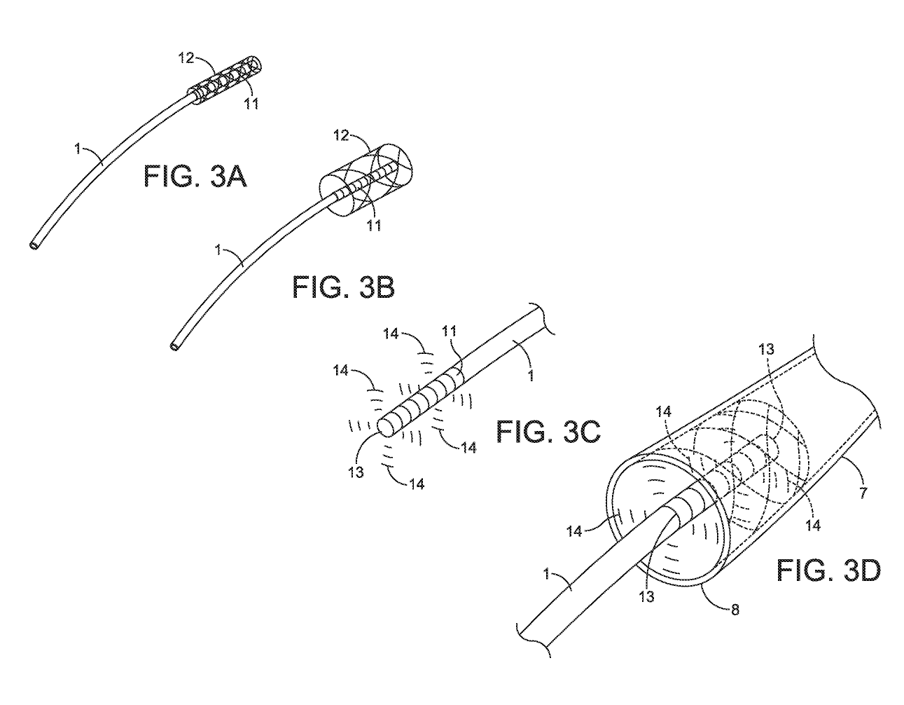

Referring to FIGS. 3A-D, a catheter has a tip in the form of an omnidirectional ablating element, such as ultrasound, microwave or laser. The catheter has an expandable stent that centralizes the ablating element within its core. When the stent is passed into the target blood vessel and deployed, the omnidirectional ablating element ablates the wall of the blood vessel while symmetrically positioned within it in a generally co-axial configuration.

In FIG. 3A, the inlet tube 1 of the catheter system connects to an omnidirectional ablating element 11 and seating stent 12 at its tip, in collapsed configuration.

In FIG. 3B, in one embodiment, the seating stent 12 of the catheter system embodies an expandable seating/positioning stent, thereby showing that omnidirectional ablating element 11 becomes centralized within the seating stent 12.

In FIG. 3C, which is an opposite view of the ablating element 11 as seen from one pole 13, the ablating element is shown to emit an omnidirectional 360-degree dispersion of ablating energy 14, most likely but not limited to microwave, ultrasound or laser energy.

In FIG. 3D, the inlet tube 1 of the ablation catheter system is shown leading up to the expandable seating stent 12 within coronary sinus 7. The ablating element 11 is centralized within the seating stent 12, which generally conforms to the shape of the coronary sinus, thereby allowing equal dispersion of omnidirectional 360-degree energy to ablate the wall of the coronary sinus. The surface sensors 5 facilitate the procedure. Blood flows freely within the seating stent 12 and around the centralized ablating element 11.

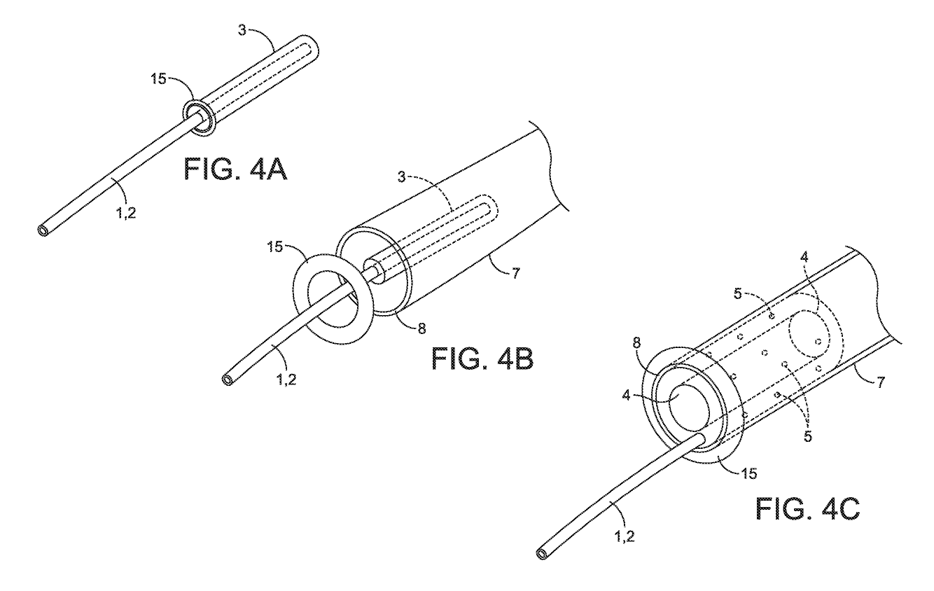

Referring to FIGS. 4A-C, a catheter has a tip in the form of both a large expandable seating balloon or stent and the ablating expandable balloon. The seating balloon is larger in surface diameter than the diameter of the blood vessel to be ablated so that it can be positioned flush with the vessel opening and allow the ablating expandable balloon to be perfectly positioned within the terminal portion of the blood vessel. Surface sensors facilitate the ablation procedure.

In FIG. 4A, the inlet tube 1 and the outlet/exhaust tube 2 of the catheter system includes two expandable elements, seating element 15 and the expandable ablating balloon or stent 3 at its distal end, which is shown in a collapsed configuration.

In FIG. 4B, the expandable element 15 is shown expanded, with the expandable element being larger that the diameter of the coronary sinus opening 8. The inlet tube 1 and the outlet/exhaust tube 2 of the catheter system are connected to collapsed expandable balloon 3 distant to the expandable seating element 15, which is advanced into coronary sinus 7 via its sinus opening 8. The expandable seating element 15, which may be a balloon and/or a stent (not shown), includes a large central core or defect to allow blood to pass through it unobstructed.

In FIG. 4C, the inlet tube 1 and the outlet/exhaust tube 2 of the ablating catheter system has been advanced so that expandable seating element 15 is positioned flush against the coronary sinus opening 8, and cannot pass through due to its larger size. The expandable ablating balloon (or stent) 3 at the leading edge of the catheter system is expanded within coronary sinus 7. Blood can pass through both expandable ablating balloon 3 and the expandable seating element 15 through the large central core 4. The surface sensors 5 facilitate the procedure. When ablation is completed the device is collapsed and withdrawn.

The phraseology and terminology used herein is for the purpose of description and should not be regarded as limiting. Any references to embodiments or elements or acts of the systems and methods herein referred to in the singular may also embrace embodiments including a plurality of these elements, and any references in plural to any embodiment or element or act herein may also embrace embodiments including only a single element. References in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. Any references to front and back, left and right, top and bottom, upper and lower, and vertical and horizontal are intended for convenience of description, not to limit the present systems and methods or their components to any one positional or spatial orientation.

Having thus described several aspects of at least one embodiment, it is to be appreciated various alterations, modifications, and improvements will readily occur to those skilled in the art. Such alterations, modifications, and improvements are intended to be part of this disclosure and are intended to be within the scope of the disclosure. Accordingly, the foregoing description and drawings are by way of example only, and the scope of the disclosure should be determined from proper construction of the appended claims, and their equivalents.

What is claimed is:

1. A device for internal circumferential ablation of a tubular vascular structure, the device comprising:
an expandable structure having a central opening extending therethrough to allow a relatively unobstructed flow of body fluid through the tubular vascular structure,
wherein the expandable structure is expandable from a collapsed configuration to an expanded configuration, the expandable structure being configured to be secured in a desired location on the tubular vascular structure,
wherein the expandable structure further is configured to emit or absorb energy to ablate tissue.

2. The device of claim 1, wherein the expandable structure is a balloon including an ablating element, the balloon being generally shaped like a torus or elongated cylinder having a channel.

3. The device of claim 2, wherein the balloon includes a surface having a plurality of sensors.

4. The device of claim 2, wherein the expandable structure is configured to apply a radial contact force on the tubular vascular structure to secure the expandable structure to the tubular vascular structure.

5. The device of claim 2, wherein the balloon is positioned at a tip of a longer catheter, and wherein the catheter is used to deliver gas or liquid to and from the balloon to accomplish ablation by releasing or absorbing energy.

6. The device of claim 2, wherein the balloon houses an array of small bore tubes to generally equally disperse gas or liquid for equal ablation along an expanded circumference of the balloon.

7. The device of claim 2, wherein the balloon is a compliant balloon that generally assumes a shape of the vascular structure within which it expanded.

8. The device of claim 2, further comprising a second expandable structure adjacent to the balloon, the second expandable structure including a larger surface area than the diameter of the balloon.

9. The device of claim 1, wherein the expandable structure includes a thermally conductive stent that conforms to a shape of the tubular vascular structure, the stent being connected to either a heating element or a cooling element to conduct energy through the stent.

10. The device of claim 9, further comprising surface sensors provided on the expandable structure.

11. The device of claim 9, further comprising a second expandable structure adjacent to the thermally conductive stent, the second expandable structure including a larger diameter than the thermally conductive stent.

12. The device of claim 1, wherein the expandable structure includes a stent that conforms to a shape of the tubular vascular structure, the stent housing and centering an ablating element in its center.

13. The device of claim 12, wherein the ablating element is omnidirectional and ablates using ultrasound, microwave, laser, or another form of energy.

14. The device of claim 12, further comprising surface sensors provided on the expandable structure.

15. A method of circumferential ablation of a terminal segment of a coronary sinus or other vascular structure using a catheter system in order to treat heart rhythm disorders and performing ablation without significant obstruction of blood flow, the method comprising:
positioning an expandable structure of the catheter system into a tubular vascular structure, the expandable structure having a central opening extending therethrough to allow a relatively unobstructed flow of body fluid through the tubular vascular structure;
securing the expandable structure in a desired location on the tubular vascular structure; and
expanding the expandable structure from a collapsed configuration to an expanded configuration.

16. The method of claim 15, wherein the expandable structure is a balloon including an ablating element, the balloon being generally shaped like a torus or elongated cylinder having a channel.

17. The method of claim 16, further comprising sensing one of voltage, temperature and pressure with wiring conducted along the catheter system.

18. The method of claim 17, wherein the balloon includes a surface having a plurality of sensors.

19. The method of claim 16, wherein securing the expandable structure includes expanding the expandable structure to apply a radial contact force.

20. The method of claim 16, further comprising delivering gas or liquid to and from a catheter in fluid communication with the balloon.
